(19) United States
(12) Patent Application Publication
(10) Pub. No.: US 2008/0058701 A1
(43) Pub. Date: Mar. 6, 2008

(54) DELIVERY DEVICE HAVING SELF-ASSEMBLING DENDRITIC POLYMERS AND METHOD OF USE THEREOF

(75) Inventor: Gregory A. Smith, Sammamish, WA (US)

Correspondence Address: SEED INTELLECTUAL PROPERTY LAW GROUP PLLC, 701 FIFTH AVE, SUITE 5400, SEATTLE, WA, 98104, US

(73) Assignee: TRANSCUTANEOUS TECHNOLOGIES INC., Tokyo (JP)

(21) Appl. No.: 11/773,352

(22) Filed: Jul. 3, 2007

Related U.S. Application Data
(60) Provisional application No. 60/818,827, filed on Jul. 5, 2006.

(51) Int. Cl. A61N 1/30

(52) U.S. Cl. 604/20; 604/306

(57) ABSTRACT

A device for delivery of one or more active agents to a biological interface includes a matrix having at least one dendritic polymer or arborol. In certain embodiments, the dendritic polymer or arborol self-assembles to form a matrix having aqueous pores or cavities to contain and allow transport of active agents or ions. In particular aspects, the device is an iontophoretic device. In certain aspects, the iontophoretic device may include an active electrode assembly having an active agent holding portion or reservoir; and a non-active electrode assembly.

### CROSS-REFERENCE TO RELATED APPLICATION

This application claims the benefit under 35 U.S.C. § 119(e) of U.S. Provisional Patent Application No. 60/818,827, filed Jul. 5, 2006, where this provisional application is incorporated herein by reference in its entirety.

### BACKGROUND

1. Field

This disclosure generally relates to the field of medical devices, and more particularly to devices for transdermal delivery of active agents such as therapeutic agents or drugs or diagnostic agents to a biological interface under the influence of electromotive force and/or current.

2. Description of the Related Art

Medical devices that employ electromotive forces are well known in the art. For example, iontophoretic delivery devices employ an electromotive force and/or current to transfer an active agent (e.g., a charged substance, an ionized compound, an ionic drug, a therapeutic, a bioactive agent, a diagnostic agent, and the like), to a biological interface (e.g., skin, mucous membrane, and the like), by using a small electrical charge applied to an iontophoretic chamber containing a similarly charged active agent and/or its vehicle.

Iontophoresis devices typically include an active electrode assembly and a counter electrode assembly, each coupled to opposite poles or terminals of a power source, for example a chemical battery or an external power station connected to the iontophoresis device via electrical leads. Each electrode assembly typically includes a respective electrode element to apply an electromotive force and/or current. Such electrode elements often comprise a sacrificial element or compound, for example silver or silver chloride. The active agent may be either cationic or anionic, and the power source may be configured to apply the appropriate voltage polarity based on the polarity of the active agent. Iontophoresis may be advantageously used to enhance or control the delivery rate of the active agent. The active agent may be stored in a reservoir such as a cavity. Alternatively, the active agent may be stored in a reservoir such as a porous structure or a gel. An ion exchange membrane may be positioned to serve as a polarity selective barrier between the active agent reservoir and the biological interface. The membrane, typically only permeable with respect to one particular type of ion (e.g., a charged active agent), prevents the back flux of oppositely charged ions from the skin or mucous membrane.

Commercial acceptance of iontophoresis devices is dependent on a variety of factors, such as cost to manufacture, shelf life, stability during storage, efficiency and/or timeliness of active agent delivery, biological capability, and/or disposal issues. Commercial acceptance of iontophoresis devices is also dependent on their versatility and ease-of-use.

The present disclosure is directed to overcoming one or more of the shortcomings set forth above and providing further related advantages.

### BRIEF SUMMARY

The present disclosure relates to a delivery device having a reservoir or membrane formed from a matrix with aqueous pores and/or cavities wherein the matrix may contain and allow transport of an active agent to a biological interface. In at least one embodiment, the matrix may be a self-assembling or self-associating matrix. In at least one embodiment, the delivery device may be a transdermal delivery device. In at least one embodiment, the delivery device may be an iontophoretic device. In at least one embodiment, the active agent may be a therapeutic or pharmaceutical active agent or drug. In at least one embodiment, the biological interface may be a skin or a mucous membrane.

The present disclosure further relates to a delivery device comprising a matrix including a dendritic polymer for delivery of an active agent to a biological interface. In at least one embodiment, the dendritic polymer may be a self-assembling or self-associating dendritic polymer.

The present disclosure further relates to a delivery device comprising a matrix including an arborol for delivery of an active agent to a biological interface. In at least one embodiment, the arborol may be a self-assembling or self-associating arborol.

In at least one embodiment, a device for delivery of one or more active agents to a biological interface comprises a matrix comprising at least one dendritic polymer or arborol and at least one active agent. In at least one embodiment, the device may be a transdermal delivery device. In at least one embodiment, the device may be an iontophoretic device. In at least one embodiment, the device may be an iontophoretic device having at least two electrode structures.

In at least one embodiment, a device for delivery of one or more active agents to a biological interface comprises a matrix comprising at least one arborol and at least one active agent.

In at least one embodiment, the arborol may be a one-directional arborol. In at least one embodiment, the one-directional arborol may be selected from a [9]-6, a [9]-8, or a [9]-10 arborol.

In at least one embodiment, the arborol may be a two-directional arborol. In at least one embodiment, the two-directional arborol may be a [m]-n-[m] arborol wherein m is the number of polar groups comprising each of two hydrophilic end regions and n is the number of carbons in a linear alkyl chain connecting the two hydrophilic end regions. In at least one embodiment, the two-directional arborol may be a [9]-n-[9] arborol wherein n is the number of carbons in a linear alkyl chain connecting two hydrophilic end regions each comprising nine hydroxyl groups. In at least one embodiment, the two-directional arborol may be a [9]-n-[9] arborol wherein n is 10-13. In one embodiment, the two-directional arborol may be a [9]-10-[9] arborol.

In at least one embodiment, the arborol may be a three-directional arborol. In at least one embodiment, the three-directional arborol may be benzene[9³].

In at least one embodiment, a device for delivery of one or more active agents to a biological interface comprises a matrix comprising at least one dendritic polymer and at least one active agent.

In at least one embodiment, the dendritic polymer may be a polyamidoamine (PAMAM) dendrimer. In at least one embodiment, the dendritic polymer may be a polypropylene imine (PPI) dendrimer. In one embodiment, the dendritic polymer may be a polyether dendrimer. In at least one embodiment, the dendritic polymer may be a phenylacetylene dendrimer.

In at least one embodiment, the matrix may be a gel matrix.

In at least one embodiment, a device for delivery of one or more active agents to a biological interface comprises a matrix comprising at least one arborol and at least one active agent wherein the arborol may be a self-assembling arborol.

In at least one embodiment, the self-assembling arborol may be a two-directional arborol. In at least one embodiment, the two-directional arborol may be a [m]-n-[m] arborol wherein m is the number of polar groups comprising each of two hydrophilic end regions and n is the number of carbons in a linear alkyl chain connecting the two hydrophilic end regions. In at least one embodiment, the two-directional arborol may be a [9]-n-[9] arborol wherein n is the number of carbons in a linear alkyl chain connecting two hydrophilic end regions each comprising nine hydroxyl groups. In at least one embodiment, the two-directional arborol may be a [9]-n-[9] arborol wherein n is 10-13. In one embodiment, the two-directional arborol may be a [9]-10-[9] arborol.

In at least one embodiment, a device for delivery of one or more active agents to a biological interface comprises a matrix comprising at least one dendritic polymer or arborol and a least one active agent wherein the active agent may be a therapeutic agent, a diagnostic agent, or a pharmaceutical drug.

In at least one embodiment, a device for delivery of one or more active agents to a biological interface comprises a matrix comprising at least one dendritic polymer or arborol and a least one active agent and further comprises at least one reservoir.

In at least one embodiment, a device for delivery of one or more active agents to a biological interface comprises a matrix comprising at least one dendritic polymer or arborol and at least one active agent wherein the matrix comprising at least one dendritic polymer or arborol forms a reservoir. In at least one embodiment, the dendritic polymer or arborol may be a self-assembling dendritic polymer or arborol. In at least one embodiment, self-assembling the dendritic polymer or arborol forms the reservoir.

In at least one embodiment, a device for delivery of one or more active agents to a biological interface comprises a matrix comprising at least one dendritic polymer or arborol and at least one active agent and further comprises at least one membrane.

In at least one embodiment, a device for delivery of one or more active agents to a biological interface comprises a matrix comprising at least one dendritic polymer or arborol and at least one active agent wherein the matrix comprising at least one dendritic polymer or arborol forms at least one membrane. In at least one embodiment, the dendritic polymer or arborol may be a self-assembling dendritic polymer or arborol. In at least one embodiment, self-assembling the dendritic polymer or arborol forms at least one membrane.

In at least one embodiment, a device for delivery of one or more active agents to a biological interface comprises a matrix comprising at least one dendritic polymer or arborol and a least one active agent wherein the dendritic polymer or arborol may comprise a charged group. In at least one embodiment, the charged group may have a net positive charge. In at least one embodiment, the charged group may have a net negative charge.

In at least one embodiment, a device for delivery of one or more active agents to a biological interface comprises a matrix comprising at least one dendritic polymer or arborol and a least one active agent and further comprises an interface-coupling medium between a surface of the device and the biological interface. In at least one embodiment, the interface-coupling medium may be an adhesive.

In at least one embodiment, a device for delivery of one or more active agents to a biological interface comprises a matrix comprising at least one dendritic polymer or arborol and an electrode structure. In at least one embodiment, the device may be a transdermal delivery device. In at least one embodiment, the device may be an iontophoretic device having at least two electrode structures. In at least one embodiment, the arborol may be a one-directional arborol. In at least one embodiment, the one-directional arborol may be selected from a [9]-6, a [9]-8, or a [9]-10 arborol. In at least one embodiment, the arborol may be a two-directional arborol. In at least one embodiment, the two-directional arborol may be a [m]-n-[m] arborol wherein m is the number of polar groups comprising each of two hydrophilic end regions and n is the number of carbons in a linear alkyl chain connecting the two hydrophilic end regions. In at least one embodiment, the two-directional arborol may be a [9]-n-[9] arborol wherein n is the number of carbons in a linear alkyl chain connecting two hydrophilic end regions each comprising nine hydroxyl groups. In at least one embodiment, the two-directional arborol may be a [9]-n-[9] arborol wherein n is 10-13. In at least one embodiment, the two-directional arborol may be a [9]-10-[9] arborol. In at least one embodiment, the arborol may be a three-directional arborol. In at least one embodiment, the three-directional arborol may be benzene[9³]. In at least one embodiment, the dendritic polymer may be a polyamidoamine (PAMAM) dendrimer. In at least one embodiment, the dendritic polymer may be a polypropylene imine (PPI) dendrimer. In at least one embodiment, the dendritic polymer may be a polyether dendrimer. In at least one embodiment, the dendritic polymer may be a phenylacetylene dendrimer. In at least one embodiment, the matrix may be a gel matrix.

In at least one embodiment, a device for delivery of one or more active agents to a biological interface comprises a matrix comprising at least one dendritic polymer or arborol and an electrode structure wherein the dendritic polymer or arborol may be a self-assembling dendritic polymer or arborol. In at least one embodiment, the self-assembling dendritic polymer or arborol may be a two-directional arborol. In at least one embodiment, the two-directional arborol may be a [m]-n-[m] arborol wherein m is the number of polar groups comprising each of two hydrophilic end regions and n is the number of carbons in a linear alkyl chain connecting the two hydrophilic end regions. In at least one embodiment, the two-directional arborol may be a [9]-n-[9] arborol wherein n is the number of carbons in a linear alkyl chain connecting two hydrophilic end regions each comprising nine hydroxyl groups. In at least one embodiment, the two-directional arborol may be a [9]-n-[9] arborol wherein n is 10-13. In at least one embodiment, the two-directional arborol may be a [9]-10-[9] arborol.

In at least one embodiment, a device for delivery of one or more active agents to a biological interface comprises a matrix comprising at least one dendritic polymer or arborol and an electrode structure wherein the active agent may be a therapeutic agent, a diagnostic agent, or a pharmaceutical drug.

In at least one embodiment, a device for delivery of one or more active agents to a biological interface comprises a matrix comprising at least one dendritic polymer or arborol and an electrode structure and further comprises at least one reservoir comprising a dendritic polymer or arborol. In at least one embodiment, the dendritic polymer or arborol may be a self-assembling dendritic polymer or arborol. In at least one embodiment, self-assembling the dendritic polymer or arborol forms the reservoir.

In at least one embodiment, a device for delivery of one or more active agents to a biological interface comprises a matrix comprising at least one dendritic polymer or arborol and an electrode structure and further comprises at least one membrane comprising a dendritic polymer or arborol. In at least one embodiment, the dendritic polymer or arborol may be a self-assembling dendritic polymer or arborol. In at least one embodiment, self-assembling the dendritic polymer or arborol forms at least one membrane. In at least one embodiment, the dendritic polymer or arborol comprises a charged group. In at least one embodiment, the charged group may have a net positive charge. In at least one embodiment, the charged group may have a net negative charge. In at least one embodiment, a device for delivery of one or more active agents to a biological interface comprises a matrix comprising at least one dendritic polymer or arborol and an electrode structure and further comprises an interface-coupling medium between a surface of the device and the biological interface. In at least one embodiment, the interface-coupling medium may be an adhesive.

In at least one embodiment, a method for making an active agent delivery device comprises placing in a portion of the device a solution or suspension of a self-associating dendritic polymer and allowing the self-associating dendritic polymer to self associate. In at least one embodiment, the portion of the device may be a reservoir. In at least one embodiment, the portion of the device may be a membrane. In at least one embodiment, the self-associating dendritic polymer may be an arborol. In at least one embodiment, an active agent delivery device may be a device made by one of these methods.

In at least one embodiment, a method for making an active agent delivery device comprises placing in a portion of the device a solution or suspension of a self-associating dendritic polymer and allowing the self-associating dendritic polymer to self associate further comprises loading an active agent into pores or cavities created by self-association of the self-associating dendritic polymer. In one embodiment, an active agent delivery device may be a device made by one of these methods.

In at least one embodiment, a transdermal delivery system is an iontophoretic system comprising an iontophoretic device. In at least one embodiment, an iontophoretic device comprises an active electrode assembly and a counter electrode assembly, each coupled to opposite poles or terminals of a power source. In such an embodiment, each electrode assembly typically includes a respective electrode element to apply an electromotive force. Such electrode elements often comprise a sacrificial element or compound, for example silver or silver chloride.

An active agent, such as a therapeutic agent, diagnostic agent, or pharmaceutical drug, may be a cation, an anion, or a mixture of such. A power source may be configured to apply the appropriate voltage polarity based on the polarity of the active agent to be transported at a particular time and/or location. Iontophoresis may be advantageously used to enhance or control the delivery rate of the active agent. The active agent may be stored in and transported from a reservoir, such as a cavity. Alternatively, the active agent may be stored in a reservoir, such as a porous structure or gel, for example comprising aqueous pores and/or cavities within a matrix that has been self-assembled from dendritic polymers or arborols. An ion-exchange membrane may be positioned to serve as a polarity selective barrier between the active agent reservoir and the biological interface, thereby preventing backward flux of the oppositely charged ions from the skin or mucous membrane(s)

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1A is a top, front view of a transdermal delivery system according to one illustrated embodiment.

FIG. 1B is a top, plan view of a transdermal delivery system according to one illustrated embodiment.

FIG. 2A is a schematic diagram of the transdermal delivery device of FIGS. 1A and 1B comprising an active electrode assembly and a counter electrode assembly according to one illustrated embodiment.

FIG. 2B is a schematic diagram of the transdermal delivery device of FIG. 2A positioned on a biological interface, with an optional outer release liner removed to expose the active agent, according to another illustrated embodiment.

### DETAILED DESCRIPTION

In the following description, certain specific details are included to provide a thorough understanding of various disclosed embodiments. One skilled in the relevant art, however, will recognize that embodiments may be practiced without one or more of these specific details, or with other methods, components, materials, etc. In other instances, well-known structures associated with electrically powered devices, including but not limited to voltage and/or current regulators, have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment,” or “an embodiment, or “in another embodiment” means that a particular referent feature, structure, or characteristic described in connection with the embodiment is included in at least one embodiment. Thus, appearance of the phrases “in one embodiment,” or “in an embodiment, or “in another embodiment” in various places throughout this specification are not necessarily all referring to the same embodiment. Furthermore, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to a transdermal delivery device including “an electrode element” includes a single electrode element, or two or more electrode elements. It should also be noted that the term “or” is generally employed in its sense including “and/or” unless the content clearly dictates otherwise.

As used herein, the term “membrane” means a boundary, layer, barrier, or material, which may or may not be permeable. The term “membrane” may further refer to an interface. Unless specified otherwise, membranes may take the form of a solid, a liquid or a gel, and may or may not have a distinct lattice, non-cross-linked structure, or cross-linked structure. Membranes may comprise dendritic polymers or arborols or gels or matrices assembled therefrom, as further described elsewhere herein.

As used herein, the term “ion selective membrane” means a membrane that is substantially selective to ions, passing certain ions while blocking passage of other ions. An ion selective membrane, for example, may take the form of a charge selective membrane, or may take the form of a semi-permeable membrane.

As used herein, the term “charge selective membrane” means a membrane that substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. A cation exchange membrane substantially permits the passage of cations and substantially blocks anions. Examples of commercially available cation exchange membranes include those available under the designators NEOSEPTA, CM-1, CM-2, CMX, CMS, and CMB from Tokuyama Co., Ltd. Conversely, an anion exchange membrane substantially permits the passage of anions and substantially blocks cations. Examples of commercially available anion exchange membranes include those available under the designators NEOSEPTA, AM-1, AM-3, AMX, AHA, ACH, and ACS, also from Tokuyama Co., Ltd.

As used herein, the term “bipolar membrane” means a membrane that is selective to two different charges or polarities. Unless specified otherwise, a bipolar membrane may take the form of a unitary membrane structure, a multiple membrane structure, or a laminate. The unitary membrane structure may include a first portion including cation ion exchange materials or groups and a second portion opposed to the first portion, including anion ion exchange materials or groups. The multiple membrane structure (e.g., two-film structure) may include a cation exchange membrane laminated or otherwise coupled to an anion exchange membrane. The cation and anion exchange membranes initially start as distinct structures, and may or may not retain their distinctiveness in the structure of the resulting bipolar membrane.

As used herein, the term “semi-permeable membrane” means a membrane that is substantially selective based on a size or molecular weight of the ion. Thus, a semi-permeable membrane substantially passes ions of a first molecular weight or size, while substantially blocking passage of ions of a second molecular weight or size, greater than the first molecular weight or size. In some embodiments, a semi-permeable membrane may permit the passage of some molecules at a first rate, and some other molecules at a second rate different from the first. In yet further embodiments, the “semi-permeable membrane” may take the form of a selectively permeable membrane allowing only certain selective molecules to pass through it.

As used herein, the term “porous membrane” means a membrane that is not substantially selective with respect to ions at issue. For example, a porous membrane is one that is not substantially selective based on polarity, and not substantially selective based on the molecular weight or size of a subject element or compound.

As used herein, the term “gel matrix” means a type of reservoir, which takes the form of a three-dimensional network, a colloidal suspension of a liquid in a solid, a semi-solid, a cross-linked gel, a non-cross-linked gel, a jelly-like state, and the like. In some embodiments, the gel matrix may result from a three-dimensional network of entangled macromolecules (e.g., cylindrical micelles). In some embodiments, a gel matrix may include hydrogels, organogels, and the like. Hydrogels refer to three-dimensional networks of, for example, cross-linked hydrophilic polymers in the form of a gel and substantially composed of water. Hydrogels may have a net positive or net negative charge, or may be neutral.

In certain embodiments, the gel matrix comprises dendritic polymers. Dendritic polymers, also termed dendrimers or hyperbranched polymers, have well-defined structures. Dendrimers are highly branched, typically monodisperse, structures, having consistent size and form. These branched polymer molecules were first described in the early 1980's. D. A. Tomalia and his co-workers identified their ‘starburst’ structures as dendrimers (Polymer J. 17:117-132, 1985). G. R. Newkome and his co-workers identified their ‘cascade molecules’ as arborols (J. Org. Chem. 50:2003-2006, 1985). Dendritic polymers are generally prepared by either divergent or convergent synthetic procedures. In many embodiments, dendrimers consist of three major structural components: core, branches, and end groups. In certain embodiments, dendrimers are described as having a star-like or starburst structure, where the branching occurs from a central core.

Dendrimers are synthesized in an iterative sequence of reactions, in which each additional iteration leads to a higher generation dendrimers. Synthesis of dendrimers is an example of controlled hierarchical synthesis, each step leading to a new generation and increasing (e.g., doubling) the number of end groups. The synthetic process allows size, composition and number of end groups to be controlled relatively easily and efficiently. Synthesis may be carried out in a divergent or a convergent manner. Both synthetic procedures are based on the repetition of a sequence of reactions, with each sequence creating a new dendrimer generation. Divergent synthesis, first used by F. Vögtle and co-workers, is based on the successive attachment of branching units to a core molecule. Each new reiterative reaction is characterized by the generation of an exponentially increasing number of functional groups on the periphery of the dendrimer. Convergent synthesis, first used by J. M. J. Frechet and coworkers for the synthesis of poly(aryl ether) dendrimers, is based on synthesis of dendrimeric fragments, followed by subsequent addition of these fragments to the core. Both synthetic procedures lead to dendrimeric structures in which the branching portions surround a central focal point.

In certain embodiments, dendritic polymers may self-organize or self-assemble into certain forms, for example, hydrogels or gel matrices. In certain such embodiments, the dendritic polymer may be amphiphilic, comprising hydrophobic (water-fearing) and hydrophilic (water-loving) regions. In a solvent, such amphiphilic molecules orient their domain having the highest affinity for the solvent toward that solvent, while the other portion of the molecule is oriented so as to avoid contact with the solvent. In certain embodiments, spontaneous self-assembly may thus occur by virtue of one part of the polymer molecule being repelled by the surrounding medium and another part of the molecule being attracted by it. In certain embodiments, for example, the solvent may be an aqueous solvent. In such embodiments, the amphiphilic dendritic polymers may self-organize or self-assemble with the hydrophobic regions being directed to and associating with one another and the hydrophilic regions being directed outward toward and associating with the aqueous solvent.

In certain embodiments, the dendritic polymers may be dumbbell-shaped (bolaform) amphiphilic molecules. Such polymers, termed arborols, were first described and synthesized by G. R. Newkome and coworkers (J. Org. Chem. 50:2003-2006, 1985; J. Chem. Soc.—Chem. Commun. 752-753, 1986).

In certain embodiments, arborols are small molecule dendrimers that can self-assemble to form hydrogels. In certain other embodiments, arborols can form fibers. In certain embodiments, arborols may be, but are not limited to, one-directional, two-directional, or three-directional. In at least one embodiment, one-directional arborols comprise a hydrophilic end region and a hydrophobic end region. In at least one other embodiment, two-directional arborols comprise two hydrophilic end regions, connected by a hydrophobic region. In at least one further embodiment, three-directional arborols comprise three hydrophilic end regions connected via a hydrophobic central core region.

In certain embodiments, one-directional arborols may include, but are not limited to, [9]-6, [9]-8, or [9]-10 arborols. These refer to arborols wherein an hydrophilic end region comprises 9 hydroxyl groups and an hydrophobic end region comprises a 6-, 8-, or 10-carbon linear alkyl chain. In one embodiment, a [9]-10 one-directional arborol may be represented as follows: 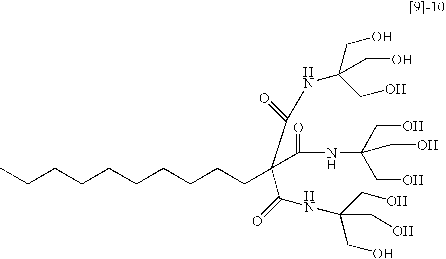
In at least one embodiment, one-directional arborols may self-assemble to form fibers when placed in a mixture of polar organic solvents and water, for example, methanol and water.

In certain embodiments, two-directional arborols may be identified as [m]-n-[m] arborols wherein m indicates the number of polar groups comprising each of two hydrophilic end regions and n represents the number of carbons in a linear alkyl chain connecting the two hydrophilic end regions. In certain aspects, the polar groups may be hydroxyl groups. In at least some aspects, m may vary between 6 and 9, and m between 7 and 13. In certain such embodiments, two-directional arborols may include, but are not limited to, structures identified as [9]-n-[9], which represent arborols in which a linear alkyl chain of n carbons connects two hydrophilic end regions each having 9 hydroxyl groups. In certain such embodiments, n may vary between 10 and 13. In one embodiment, a [9]-10-[9] two-directional arborol may be represented as follows: 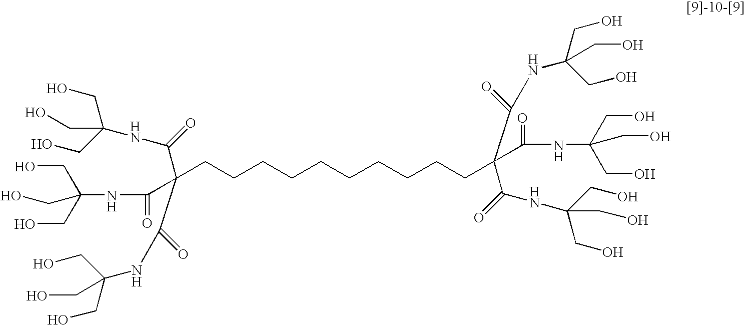
In certain embodiments, such two-dimensional arborols may self-assemble to form thermally reversible aqueous gels, wherein the arborols can dissolve in warm water and associate upon cooling to form a gel comprising extended fibrillar structures and further comprising pores and/or cavities. In one embodiment, for example, a gel may form by self-assembly of two-directional [9]-10-[9] arborols from a dilute (0.1%) aqueous solution. In certain embodiments, the extent of self-assembly of two-directional arborols to form a gel may be controlled as necessary by addition of one-directional arborols to inhibit the extent of self-association of the two-directional arborols. In one embodiment, a one-directional [9]-6 arborol may be added to a solution of two-directional [9]-10-[9] arborol to limit association of the [9]-10-[9] arborol and control the extent of the gel formed.

In certain embodiments, three-directional arborols may include, but are not limited to, arborols wherein hydrophilic branched end regions extend from a cyclic aliphatic or aromatic carbon ring structure. In one embodiment, a benzene[9³] arborol may be represented as follows:

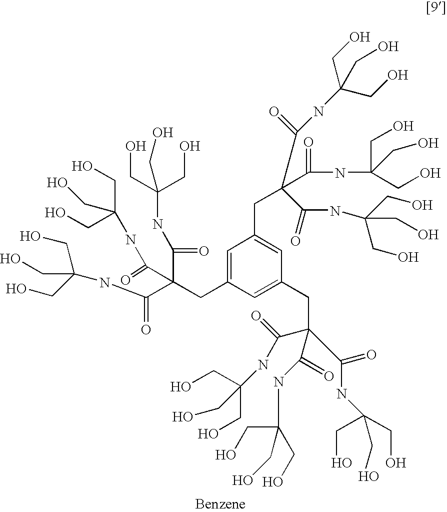

In certain embodiments, one-directional and two-directional arborols may be prepared by a two-step nucleophilic substitution-amidation procedure. In certain such embodiments, the esters may be prepared by the reaction of the appropriate bromoalkane with NaC(CO₂Et)₃ in dry dimethylformamide at 90° C. overnight.

In certain embodiments, one-dimensional arborols may be prepared by adding an appropriate bromoalkane to a stirred solution of NaC(CO₂Et)₃ in dimethylformamide at 90° C. After reaction for 12 hours, the solution is cooled and toluene is added. The solution is washed with saturated NaHCO₃, dried over anhydrous MgSO₄, concentrated in vacuo, and then dried under high vacuum in an oven at 40° C. overnight to yield the triethyl alkanehexacarboxylate ester. The ester and tris-hydroxymethylaminomethane are then dissolved in Me₂SO₄ and stirred for 4-5 days at room temperature over extra anhydrous K₂CO₃. The mixture is then filtered, poured into water to precipitate the product, centrifuged, washed twice with water, and dried under high vacuum in an oven at 40° C. overnight to yield the one-directional arborol.

In certain embodiments, two-dimensional arborols may be prepared by adding an appropriate bromoalkane to a stirred solution of NaC(CO₂Et)₃ in DMF at 90° C. After reaction for 12 hours, the solution is cooled and toluene is added. The solution is washed with saturated NaHCO₃, dried over anhydrous MgSO₄, concentrated in vacuo, and then dried under high vacuum in an oven at 40° C. overnight to yield the hexaethyl alkanehexacarboxylate ester. The ester and tris-hydroxymethylaminomethane are then dissolved in Me₂SO₄ and stirred for 4-5 days at room temperature over extra anhydrous K₂CO₃. The mixture is then filtered and evaporated in vacuo. The gel residue is dissolved in water, and the solid product is precipitated by slow addition of acetone. The product is dried under high vacuum in an oven at 40° C. overnight to yield the two-directional arborol.

In certain embodiments, dendrimers may be polyamidoamine (PAMAM) dendrimers, having tertiary amines as branching points, as described by Tomalia, D. A. et al. (Polymer J. 17:117-132, 1985; Angew. Chem., Int. Ed. 29:138-175, 1990). In some embodiments, PAMAM dendrimers may be synthesized by divergent methods, beginning with ammonia or ethylenediamine as the core initiator reagent. In certain such embodiments, the PAMAM dendrimers are constructed by an iterative sequence of reactions consisting of (a) a double Michael addition of methyl acrylate to a primary amino group followed by (b) amidation of the resulting carbomethoxy intermediate with a large excess of ethylenediamine.

In certain embodiments, dendrimers may be polypropylene imine (PPI) dendrimers. In some embodiments, PPI dendrimers may be synthesized by divergent methods, starting from 1,4-diaminobutane. In certain such embodiments, the PPI dendrimers are constructed by an iterative sequence of reactions consisting of (a) a double Michael addition of acrylonitrile to the primary amino groups of the 1,4-diaminobutane, followed by (b) hydrogenation under pressure in the presence of Raney cobalt.

In certain embodiments, aromatic polyether dendrimers and phenylacetylene dendrimers may be prepared by convergent synthetic methods.

Structural forms comprising dendrimers include, but are not limited to, gels, films, membranes, and coatings. In certain embodiments, by virtue of the dendrimeric chemistry, the structures have pores or cavities. The physicochemical character of the surfaces of these pores or cavities may be controlled by selection of the chemical nature of the components from which the dendrimers are synthesized. In certain embodiments, the end groups of the dendrimers comprise hydroxyl groups. In other embodiments, the end groups may further comprise anion-exchange groups. In yet other embodiments, the end groups may comprise cation-exchange groups. The dendrimer-containing matrix may thus include not only pores or cavities to store and serve as passageways for movement of ionic or non-ionic solutes, including active agents, but may also serve as an ion-exchange medium for use as described elsewhere herein. In certain embodiments, ion-exchange groups within the pores or cavities may serve as binding sites for active agents or other materials to be delivered iontophoretically to a biological interface.

In certain embodiments, dendritic polymers or arborols may be advantageously employed in the manufacture and use of transdermal delivery systems and devices, including iontophoretic systems and devices. In certain embodiments, for example, a matrix formed from dendritic polymers or arborols may form a reservoir or membrane structure within a transdermal system or device. In certain embodiments, a matrix so formed may comprise an active agent and/or an electrolyte composition. In certain embodiments, self-associating or self-assembling dendritic polymers or arborols may be particularly advantageously employed in the manufacture and use of transdermal delivery devices, including iontophoretic systems and devices.

Dendritic polymers and arborols are further described in U.S. Pat. Nos. 4,289,872; 4,410,688; 4,507,466; 4,558,120; 4,568,737; 4,587,329; 4,690,985; 4,737,550; 4,857,599; 5,041,516; 5,136,096; 5,154,853; 5,206,410; 5,210,309; 5,338,532; 5,376,690; 5,393,795; 5,422,379; 5,516,810; 5,527,524; 5,585,457; 5,631,329; 5,650,101; 5,703,271; 5,714,166; 5,731,095; 5,773,527; 5,773,551; 5,863,919; 5,886,126; 5,886,127; 5,919,442; 6,043,336; 6,130,209; 6,177,414; 6,228,978; 6,300,424; 6,312,679; 6,399,717; 6,566,409; 6,632,889; 6,635,720; 6,664,315; 6,995,234; 7,005,124; 7,078,461; and 7,183,426; all of which are incorporated herein in their entirety. Further description and discussion of dendritic polymers and arborols may be found in Newkome, G. R., et al. (1986) J. Chem. Soc., Chem. Commun. 752-753; Newkome, G. R., et al. (1986) J. Amer. Chem. Soc. 108, 849-850; Newkome, G. R., and Baker, G. R. (1986) Org. Prep. Proc. Intl. 18,117; Newkome, G. R., et al. (1990) J. Amer. Chem. Soc. 112, 8458-8465; Newkome, G. R., et al. (1996) J. Chem. Soc., Chem. Commun. 2737-2738; Deb, S. K., et al. (1997) J. Amer. Chem. Soc. 119, 9079-9080; Yu, K. H., et al. (1997) J. Polymer Sci. Part B, Polymer Physics 35, 2787-2793; Newkome, G. R., et al. (1998) Angew. Chem. Int. Ed. 37, 307-310; Newkome, G. R., et al. (1998) Des. Monom. Polym. 1, 3-14; Newkome, G. R., et al. (1999) Angew. Chem. Int. Ed. 38, 3717-3721; Newkome, G. R., et al. (1999) Biotech. Bioeng. (Combinatorial Chemistry) 61, 243-253; and Klajnert, B., and Bryszewska, M. (2001) Acta Biochim. Polonica 48, 199-208.

As used herein, the term “reservoir” means any form or mechanism to retain an element, compound, pharmaceutical composition, active agent, diagnostic agent, and the like, in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound. Reservoirs may comprise dendritic polymers or arborols or gels or matrices assembled therefrom, as further described elsewhere herein. Typically, a reservoir serves to retain a biologically active agent prior to the discharge of such agent by electromotive force and/or current into the biological interface. A reservoir may also retain an electrolyte solution.

As used herein, the term “active agent” refers to a compound, molecule, or treatment that elicits a biological response from any host, animal, vertebrate, or invertebrate, including, for example, fish, mammals, amphibians, reptiles, birds, and humans. Examples of active agents include therapeutic agents, pharmaceutical agents, pharmaceuticals (e.g., a drug, a therapeutic compound, pharmaceutical salts, and the like), non-pharmaceuticals (e.g., a cosmetic substance, and the like), a diagnostic agent, a vaccine, an immunological agent, a local or general anesthetic or painkiller, an antigen, a protein or peptide, such as insulin, a chemotherapeutic agent, and an anti-tumor agent.

In some embodiments, the term “active agent” refers to the active agent as well as to its pharmacologically active salts, pharmaceutically acceptable salts, prodrugs, metabolites, analogs, and the like. In some further embodiments, the active agent includes at least one ionic, cationic, ionizable, and/or neutral therapeutic drug, and/or pharmaceutically acceptable salts thereof. In yet other embodiments, the active agent may include one or more “cationic active agents” that are positively charged and/or are capable of forming positive charges in aqueous media. For example, many biologically active agents have functional groups that are readily convertible to a positive ion or can dissociate into a positively charged ion and a counter ion in an aqueous medium. For instance, an active agent having an amino group can typically take the form of an ammonium salt in solid state and dissociate into a free ammonium ion (NH₄⁺) in an aqueous medium of appropriate pH. Other active agents may have functional groups that are readily convertible to a negative ion or can dissociate into a negatively charged ion and a counter ion in an aqueous medium. Yet other active agents may be polarized or polarizable, that is, exhibiting a polarity at one portion relative to another portion.

The term “active agent” may also refer to electrically neutral agents, molecules, or compounds capable of being delivered via electro-osmotic flow. The electrically neutral agents are typically carried by the flow of, for example, a solvent during electrophoresis. Selection of the suitable active agents is therefore within the knowledge of one skilled in the relevant art.

In some embodiments, one or more active agents may be selected from analgesics, anesthetics, anesthetics vaccines, antibiotics, adjuvants, immunological adjuvants, immunogens, tolerogens, allergens, toll-like receptor agonists, toll-like receptor antagonists, immuno-adjuvants, immuno-modulators, immuno-response agents, immuno-stimulators, specific immuno-stimulators, non-specific immuno-stimulators, and immuno-suppressants, or combinations thereof.

Non-limiting examples of such active agents include lidocaine, articaine, and others of the -caine class; morphine, hydromorphone, fentanyl, oxycodone, hydrocodone, buprenorphine, methadone, and similar opioid agonists; sumatriptan succinate, zolmitriptan, naratriptan HCl, rizatriptan benzoate, almotriptan malate, frovatriptan succinate and other 5-hydroxytryptamine1 receptor subtype agonists; resiquimod, imiquidmod, and similar TLR 7 and TLR 8 agonists and antagonists; domperidone, granisetron hydrochloride, ondansetron and other such anti-emetic drugs; zolpidem tartrate and similar sleep inducing agents; L-DOPA and other anti-Parkinson's medications; aripiprazole, olanzapine, quetiapine, risperidone, clozapine, and ziprasidone, as well as other neuroleptica; diabetes drugs such as exenatide; as well as peptides and proteins for treatment of obesity and other maladies.

Further non-limiting examples of active agents include ambucaine, amethocaine, isobutyl p-aminobenzoate, amolanone, amoxecaine, amylocaine, aptocaine, azacaine, bencaine, benoxinate, benzocaine, N,N-dimethylalanylbenzocaine, N,N-dimethylglycylbenzocaine, glycylbenzocaine, beta-adrenoceptor antagonists betoxycaine, bumecaine, bupivicaine, levobupivicaine, butacaine, butamben, butanilicaine, butethamine, butoxycaine, metabutoxycaine, carbizocaine, carticaine, centbucridine, cepacaine, cetacaine, chloroprocaine, cocaethylene, cocaine, pseudococaine, cyclomethycaine, dibucaine, dimethisoquin, dimethocaine, diperodon, dyclonine, ecognine, ecogonidine, ethyl aminobenzoate, etidocaine, euprocin, fenalcomine, fomocaine, heptacaine, hexacaine, hexocaine, hexylcaine, ketocaine, leucinocaine, levoxadrol, lignocaine, lotucaine, marcaine, mepivacaine, metacaine, methyl chloride, myrtecaine, naepaine, octacaine, orthocaine, oxethazaine, parenthoxycaine, pentacaine, phenacine, phenol, piperocaine, piridocaine, polidocanol, polycaine, prilocaine, pramoxine, procaine (NOVOCAINE®), hydroxyprocaine, propanocaine, proparacaine, propipocaine, propoxycaine, pyrrocaine, quatacaine, rhinocaine, risocaine, rodocaine, ropivacaine, salicyl alcohol, tetracaine, hydroxytetracaine, tolycaine, trapencaine, tricaine, trimecaine tropacocaine, zolamine, a pharmaceutically acceptable salt thereof, and mixtures thereof.

As used herein, the term “subject” generally refers to any host, animal, vertebrate, or invertebrate, and includes fish, mammals, amphibians, reptiles, birds, and particularly humans.

As used herein, the term “agonist” refers to a compound that can combine with a receptor (e.g., an opioid receptor, toll-like receptor, and the like) to produce a cellular response. An agonist may be a ligand that directly binds to the receptor. Alternatively, an agonist may combine with a receptor indirectly by forming a complex with another molecule that directly binds the receptor, or otherwise results in the modification of a compound so that it directly binds to the receptor.

As used herein, the term “antagonist” refers to a compound that can combine with a receptor (e.g., an opioid receptor, a toll-like receptor, and the like) to inhibit a cellular response. An antagonist may be a ligand that directly binds to the receptor. Alternatively, an antagonist may combine with a receptor indirectly by forming a complex with another molecule that directly binds to the receptor, or otherwise results in the modification of a compound so that it directly binds to the receptor.

As used herein, the term “effective amount” or “therapeutically effective amount” includes an amount effective at dosages and for periods of time necessary, to achieve the desired result. The effective amount of a composition containing a pharmaceutical agent may vary according to factors such as the disease state, age, gender, and weight of the subject.

As used herein, the term “analgesic” refers to an agent that lessens, alleviates, reduces, relieves, or extinguishes a neural sensation in an area of a subject's body. In some embodiments, the neural sensation relates to pain, in other aspects the neural sensation relates to discomfort, itching, burning, irritation, tingling, “crawling,” tension, temperature fluctuations (such as fever), inflammation, aching, or other neural sensations.

As used herein, the term “anesthetic” refers to an agent that produces a reversible loss of sensation in an area of a subject's body. In some embodiments, the anesthetic is considered to be a “local anesthetic” in that it produces a loss of sensation only in one particular area of a subject's body.

As one skilled in the relevant art would recognize, some agents may act as both an analgesic and an anesthetic, depending on the circumstances and other variables including but not limited to dosage, method of delivery, medical condition or treatment, and an individual subject's genetic makeup. Additionally, agents that are typically used for other purposes may possess local anesthetic or membrane stabilizing properties under certain circumstances or under particular conditions.

As used herein, the term “immunogen” refers to any agent that elicits an immune response. Examples of an immunogen include but are not limited to natural or synthetic (including modified) peptides, proteins, lipids, oligonucleotides (RNA, DNA, etc.), chemicals, or other agents.

As used herein, the term “allergen” refers to any agent that elicits an allergic response. Some examples of allergens include but are not limited to chemicals and plants, drugs (such as antibiotics, serums), foods (such as milk, wheat, eggs, etc), bacteria, viruses, other parasites, inhalants (dust, pollen, perfume, smoke), and/or physical agents (heat, light, friction, radiation). As used herein, an allergen may be an immunogen.

As used herein, the term “adjuvant” and any derivation thereof refers to an agent that modifies the effect of another agent while having few, if any, direct effects when given by itself. For example, an adjuvant may increase the potency or efficacy of a pharmaceutical, or an adjuvant may alter or affect an immune response.

As used herein, the term “polypeptide” encompasses amino acid chains of any length, including full-length proteins, wherein the amino acids are linked by covalent peptide bonds.

As used herein, the term “opioid” generally refers to any agent that binds to and/or interacts with opioid receptors. Among the opioid classes examples include endogenous opioid peptides, opium alkaloids (e.g., morphine, codeine, and the like), semi-synthetic opioids (e.g., heroin, oxycodone and the like), synthetic opioids (e.g., buprenorphinemeperidine, fentanyl, morphinan, benzomorphan derivatives, and the like), as well as opioids that have structures unrelated to the opium alkaloids (e.g., pethidine, methadone, and the like).

As used herein, the terms “vehicle,” “carrier,” “pharmaceutical vehicle,” “pharmaceutical carrier,” “pharmaceutically acceptable vehicle,” or “pharmaceutically acceptable carrier” may be used interchangeably, and refer to pharmaceutically acceptable solid or liquid, diluting or encapsulating, filling or carrying agents, which are usually employed in the pharmaceutical industry for making pharmaceutical compositions. Examples of vehicles include any liquid, gel, salve, cream, solvent, diluent, fluid ointment base, vesicle, liposome, nisome, ethasomes, transfersome, virosome, non-ionic surfactant vesicle, phospholipid surfactant vesicle, micelle, and the like, that is suitable for use in contacting a subject.

In some embodiments, the pharmaceutical vehicle may refer to a composition that includes and/or delivers a pharmacologically active agent, but is generally considered to be otherwise pharmacologically inactive. In some other embodiments, the pharmaceutical vehicle may have some therapeutic effect when applied to a site such as a mucous membrane or skin, by providing, for example, protection to the site of application from conditions such as injury, further injury, or exposure to elements. Accordingly, in some embodiments, the pharmaceutical vehicle may be used for protection without a pharmacological agent in the formulation.

As used herein, “in conjunction with,” and any derivations thereof, refers to administration of an active agent, vehicle, carrier, and the like, simultaneously with, prior to, or subsequent to administration of a further active agent, vehicle, carrier, and the like.

The headings provided herein are for convenience only and do not interpret the scope or meaning of the embodiments.

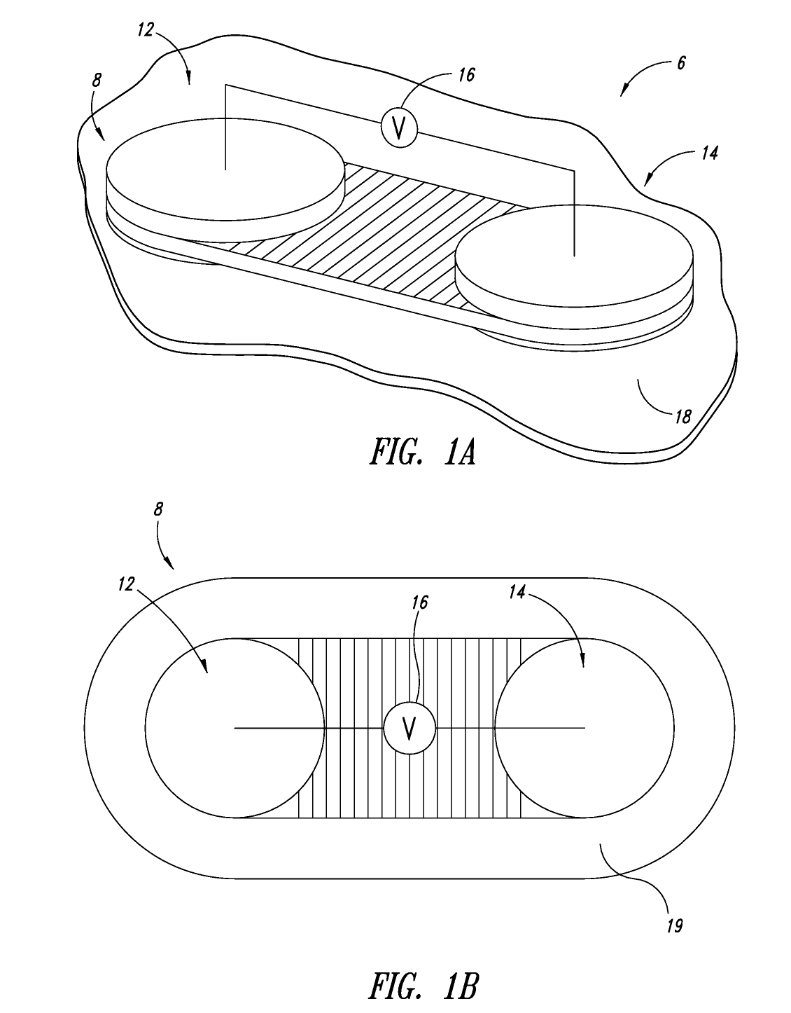

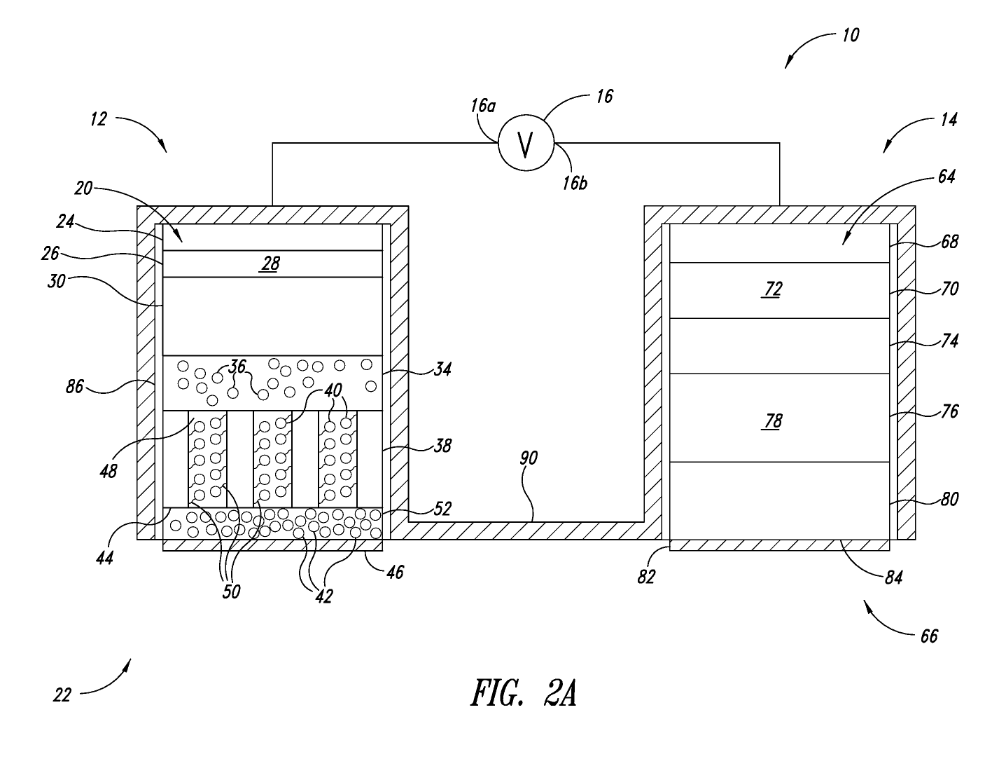

FIGS. 1A and 1B show an exemplary transdermal delivery system 10 for delivering one or more active agents to a subject. The transdermal deliver system 10 includes a transdermal delivery device 8, including active and counter electrode assemblies 12, 14, respectively, and a power source 16. In some embodiments, the power source 16 may take the form of a portable power source. The overall shape of the transdermal delivery device may take any of a variety of geometric forms.

In some embodiments, the active electrode assembly 12 may take the form of a positive electrode assembly, and the counter electrode assembly 14 may take the form of a negative electrode assembly. Alternatively, the active electrode assembly 12 may take the form of a negative assembly, and the counter electrode assembly 14 may take the form of a positive electrode assembly. The active and counter electrode assemblies are electrically coupleable to the power source 16 to supply an active agent contained in the active electrode assembly 12, via iontophoresis, to a biological interface 18, (e.g., a portion of skin or mucous membrane via iontophoresis, according to one illustrated embodiment).

The transdermal delivery device 8 may optionally include a backing 19. In some embodiments, the backing 19 encases the transdermal delivery device 8. In some other embodiments, the backing 19 physically couples the transdermal delivery device 8 to a biological interface of a subject. In some embodiments, the transdermal delivery device 8 is configured to provide transdermal delivery of one or more therapeutic active agents to a biological interface of a subject.

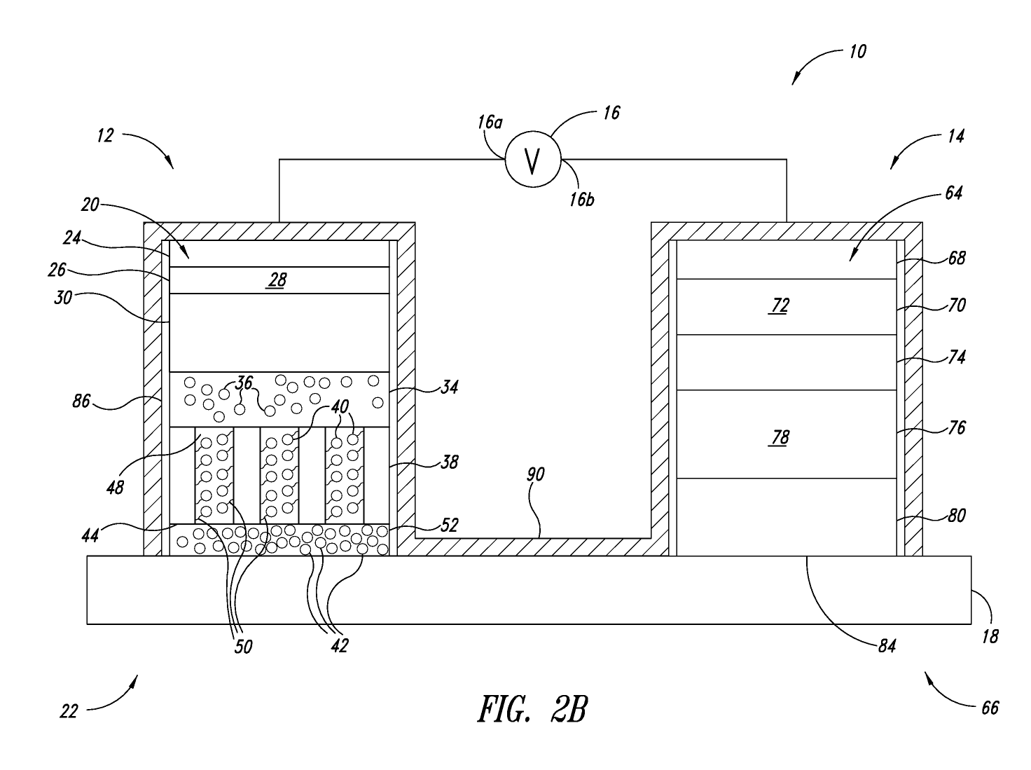

As shown in FIGS. 2A and 2B, the active electrode assembly 12 may further comprise, from an interior 20 to an exterior 22 of the active electrode assembly 12: an active electrode element 24, an electrolyte reservoir 26 storing an electrolyte 28, an inner ion selective membrane 30, one or more inner active agent reservoirs 34 (which may comprise a dendritic polymer or arborol or gels or matrices assembled therefrom, as further described elsewhere herein), storing one or more active agents 36, an optional outermost ion selective membrane 38 that optionally caches additional active agents 40, and an optional further active agent 42 carried by an outer surface 44 of the outermost ion selective membrane 38. Each of the above elements or structures will be discussed in detail below.

The active electrode assembly 12 may comprise an optional inner sealing liner (not shown) between two layers of the active electrode assembly 12, for example, between the inner ion selective membrane 30 and the inner active agent reservoir 34. The inner sealing liner, if present, would be removed prior to application of the iontophoretic device to the biological interface 18. The active electrode assembly 12 may further comprise an optional outer release liner 46.

In some embodiments, the one or more active agent reservoirs 34 are loadable with a vehicle and/or pharmaceutical composition for transporting, delivering, encapsulating, and/or carrying the one or more active agents 36, 40, 42. In some embodiments, the vehicle and/or pharmaceutical composition may comprise a dendrimer or arborol, as described elsewhere herein. In some embodiments, wherein the vehicle and/or pharmaceutical composition comprises a dendrimer, the dendrimer may be self-assembling. In some embodiments, the pharmaceutical composition includes a therapeutically effective one or more active agents 36, 40, 42.

The active electrode element 24 is electrically coupleable via a first pole 16a to the power source 16 and is positioned in the active electrode assembly 12 to apply an electromotive force to transport the active agent 36, 40, 42 via various other components of the active electrode assembly 12. Under ordinary use, the magnitude of the applied electromotive force is generally that required to deliver the one or more active agents according to a therapeutically effective dosage protocol. In some embodiments, the magnitude is selected such that it meets or exceeds the ordinary use operating electrochemical potential of the transdermal delivery system 10. The active electrode element 24 is operable to provide an electromotive force for driving a pharmaceutical composition comprising one or more active agents 36, 40, 42 from the one or more active agent reservoirs 34 to the biological interface 18 of the subject.

The active electrode element 24 may take a variety of forms. In one embodiment, the active electrode element 24 may advantageously take the form of a carbon-based active electrode element. Such may comprise multiple layers, for example a polymer matrix comprising carbon and a conductive sheet comprising carbon fiber or carbon fiber paper, such as that described in commonly assigned pending Japanese patent application 2004/317317, filed Oct. 29, 2004. The carbon-based electrodes are inert electrodes in that they do not themselves undergo or participate in electrochemical reactions. Thus, an inert electrode distributes current through the oxidation or reduction of a chemical species capable of accepting or donating an electron at the potential applied to the system (e.g., generating ions by either reduction or oxidation of water). Additional examples of inert electrodes include stainless steel, gold, platinum, capacitive carbon, or graphite.

Alternatively, an active electrode of sacrificial conductive material, such as a chemical compound or amalgam, may also be used. A sacrificial electrode does not cause electrolysis of water, but would itself be oxidized or reduced. Typically, for an anode a metal/metal salt may be employed. In such case, the metal would oxidize to metal ions, which would then be precipitated as an insoluble salt. An example of such an anode includes an Ag/AgCl electrode. The reverse reaction takes place at the cathode in which the metal ion is reduced and the corresponding anion is released from the surface of the electrode. The electrolyte reservoir may comprise a dendritic polymer or arborol, or gels or matrices assembled therefrom, as further described elsewhere herein.

The electrolyte reservoir 26 may take a variety of forms including any structure capable of retaining electrolyte 28, and, in some embodiments, may even be the electrolyte 28 itself, for example, where the electrolyte 28 is in a gel, semi-solid or solid form. For example, the electrolyte reservoir 26 may take the form of a pouch or other receptacle, or a membrane with pores, cavities or interstices, particularly where the electrolyte 28 is a liquid.

In one embodiment, the electrolyte 28 comprises ionic or ionizable components in an aqueous medium, which can act to conduct current towards or away from the active electrode element. Suitable electrolytes include, for example, aqueous solutions of salts. Preferably, the electrolyte 28 includes salts of physiological ions, such as sodium, potassium, chloride, and phosphate. In some embodiments, the one or more electrolyte reservoirs 24 include an electrolyte 28 comprising at least one biologically compatible anti-oxidant selected from ascorbate, fumarate, lactate, and malate, or salts thereof.

Once an electrical potential is applied, when an inert electrode element is in use, water is electrolyzed at both the active and counter electrode assemblies. In certain embodiments, such as when the active electrode assembly is an anode, water is oxidized. As a result, oxygen is removed from water while protons (H⁺) are produced. In one embodiment, the electrolyte 28 may further comprise an anti-oxidant. In some embodiments, the anti-oxidant is selected from anti-oxidants that have a lower potential than that of, for example, water. In such embodiments, the selected anti-oxidant is consumed rather than having the hydrolysis of water occur. In some further embodiments, an oxidized form of the anti-oxidant is used at the anode. Certain examples of biologically compatible anti-oxidants include, but are not limited to, ascorbic acid (vitamin C), tocopherol (vitamin E), or sodium citrate.

As noted above, the electrolyte 28 may take the form of an aqueous solution housed within a reservoir 26, or may take the form of a dispersion in a hydrogel or hydrophilic polymer capable of retaining substantial amount of water. For instance, a suitable electrolyte may take the form of a solution of 0.5M disodium fumarate: 0.5M polyacrylic acid: 0.15M anti-oxidant.

The inner ion selective membrane 30 is generally positioned to separate the electrolyte 28 and the inner active agent reservoir 34, if such a membrane is included within the device. The inner ion selective membrane 30 may take the form of a charge selective membrane. For example, when the active agent 36, 40, 42 comprises a cationic active agent, the inner ion selective membrane 30 may take the form of an anion exchange membrane, selective to substantially pass anions and substantially block cations. The inner ion selective membrane 30 may advantageously prevent transfer of undesirable elements or compounds between the electrolyte 28 and the inner active agent reservoir 34. For example, the inner ion selective membrane 30 may prevent or inhibit the transfer of sodium (Na⁺) ions from the electrolyte 28, thereby increasing the transfer rate and/or biological compatibility of the transdermal delivery system 10.

The inner active agent reservoir 34 is generally positioned between the inner ion selective membrane 30 and the outermost ion selective membrane 38. The inner active agent reservoir 34 may take a variety of forms including any structure capable of temporarily retaining active agent 36. For example, the inner active agent reservoir 34 may take the form of a pouch or other receptacle, or a membrane with pores, cavities, or interstices, particularly where the active agent 36 is a liquid. The inner active agent reservoir 34 further may comprise a gel matrix (which may comprise a dendritic polymer or arborol or gels or matrices assembled therefrom, as further described elsewhere herein).

Optionally, an outermost ion selective membrane 38 is positioned generally opposed across the active electrode assembly 12 from the active electrode element 24. The outermost membrane 38 may, as in the embodiment illustrated in FIGS. 2A and 2B, take the form of an ion exchange membrane having pores 48 (only one called out in FIGS. 2A and 2B for sake of clarity of illustration) of the ion selective membrane 38 including ion exchange material or groups 50 (only three called out in FIGS. 2A and 2B for sake of clarity of illustration). Under the influence of an electromotive force or current, the ion exchange material or groups 50 selectively substantially passes ions of the same polarity as active agent 36, 40, while substantially blocking ions of the opposite polarity. Thus, the outermost ion exchange membrane 38 is charge selective. Where the active agent 36, 40, 42 is a cation (e.g., lidocaine), the outermost ion selective membrane 38 may take the form of a cation exchange membrane, thus allowing the passage of the cationic active agent while blocking the back flux of the anions present in the biological interface, such as skin.

The outermost ion selective membrane 38 may optionally cache active agent 40. Without being limited by theory, the ion exchange groups or material 50 temporarily retains ions of the same polarity as the polarity of the active agent in the absence of electromotive force or current and substantially releases those ions when replaced with substitutive ions of like polarity or charge under the influence of an electromotive force or current.

Alternatively, the outermost ion selective membrane 38 may take the form of a semi-permeable or microporous membrane that is selective by size. In some embodiments, such a semi-permeable membrane may advantageously cache active agent 40, for example by employing the removably releasable outer release liner 46 to retain the active agent 40 until the outer release liner 46 is removed prior to use.

The outermost ion selective membrane 38 may be optionally preloaded with the additional active agent 40, such as ionized or ionizable drugs or therapeutic or diagnostic agents and/or polarized or polarizable drugs or therapeutic or diagnostic agents. Where the outermost ion selective membrane 38 is an ion exchange membrane, a substantial amount of active agent 40 may bond to ion exchange groups 50 in the pores, cavities or interstices 48 of the outermost ion selective membrane 38.

The active agent 42 that fails to bond to the ion exchange groups of material 50 may adhere to the outer surface 44 of the outermost ion selective membrane 38 as the further active agent 42. Alternatively, or additionally, the further active agent 42 may be positively deposited on and/or adhered to at least a portion of the outer surface 44 of the outermost ion selective membrane 38, for example, by spraying, flooding, coating, electrostatically depositing, vapordepositing, and/or otherwise. In some embodiments, the further active agent 42 may sufficiently cover the outer surface 44 and/or be of sufficient thickness so as to form a distinct layer 52. In other embodiments, the further active agent 42 may not be sufficient in volume, thickness, or coverage as to constitute a layer in a conventional sense of such term.

The active agent 42 may be deposited in a variety of highly concentrated forms such as, for example, solid form, nearly saturated solution form, or gel form. If in solid form, a source of hydration may be provided, either integrated into the active electrode assembly 12, or applied from the exterior thereof just prior to use.

In some embodiments, the active agent 36, additional active agent 40, and/or further active agent 42 may be identical or similar compositions or elements. In other embodiments, the active agent 36, additional active agent 40, and/or further active agent 42 may be different compositions or elements from one another. Thus, a first type of active agent may be stored in the inner active agent reservoir 34, while a second type of active agent may be cached in the outermost ion selective membrane 38. In such embodiments, either the first type or the second type of active agent may be deposited on the outer surface 44 of the outermost ion selective membrane 38 as the further active agent 42. Alternatively, a mix of the first and the second types of active agent may be deposited on the outer surface 44 of the outermost ion selective membrane 38 as the further active agent 42. As a further alternative, a third type of active agent composition or element may be deposited on the outer surface 44 of the outermost ion selective membrane 38 as the further active agent 42. In another embodiment, a first type of active agent may be stored in the inner active agent reservoir 34 as the active agent 36 and cached in the outermost ion selective membrane 38 as the additional active agent 40, while a second type of active agent may be deposited on the outer surface 44 of the outermost ion selective membrane 38 as the further active agent 42. Typically, in embodiments where one or more different active agents are employed, the active agents 36, 40, 42 will all be of common polarity to prevent the active agents 36, 40, 42 from competing with one another. Other combinations are possible.

In any of the above embodiments, reservoirs or membranes may optionally comprise a dendritic polymer or arborol or gels or matrices assembled therefrom, as further described elsewhere herein.

The outer release liner 46 may generally be positioned overlying or covering further active agent 42 carried by the outer surface 44 of the outermost ion selective membrane 38. The outer release liner 46 may protect the further active agent 42 and/or outermost ion selective membrane 38 during storage, prior to application of an electromotive force or current. The outer release liner 46 may be a selectively releasable liner made of waterproof material, such as release liners commonly associated with pressure sensitive adhesives. Note that the inner release liner 46 is shown in place in FIG. 2A and removed in FIG. 2B.

An interface-coupling medium (not shown) may be employed between the electrode assembly and the biological interface 18. The interface-coupling medium may take, for example, the form of an adhesive and/or gel. The gel may take the form of, for example, a hydrating gel. Selection of a suitable bioadhesive gel is within the knowledge of one skilled in the relevant art.

In the embodiment illustrated in FIGS. 2A and 2B, the counter electrode assembly 14 comprises, from an interior 64 to an exterior 66 of the counter electrode assembly 14: a counter electrode element 68, an electrolyte reservoir 70 (which may comprise a dendritic polymer or arborol or gels or matrices assembled therefrom, as further described elsewhere herein) storing an electrolyte 72, an inner ion selective membrane 74, an optional buffer reservoir 76 (which may comprise a dendritic polymer or arborol or gels or matrices assembled therefrom, as further described elsewhere herein) storing buffer material 78, an optional outermost ion selective membrane 80, and an optional outer release liner 82. Ion-exchange or ion-selective membranes may optionally comprise a dendritic polymer or arborol or gels or matrices assembled therefrom, as further described elsewhere herein.

The counter electrode element 68 is electrically coupleable via a second pole 16b of the power source 16, the second pole 16b having an opposite polarity to the first pole 16a. In one embodiment, the counter electrode element 68 is an inert electrode. For example, the counter electrode element 68 may take the form of the carbon-based electrode element discussed above.

The electrolyte reservoir 70 may take a variety of forms including any structure capable of retaining electrolyte 72, and, in some embodiments, may even be the electrolyte 72 itself, for example, where the electrolyte 72 is in a gel, semi-solid or solid form. For example, the electrolyte reservoir 70 may take the form of a pouch or other receptacle, or a membrane with pores, cavities, or interstices, particularly where the electrolyte 72 is a liquid.

The electrolyte 72 is generally positioned between the counter electrode element 68 and the outermost ion selective membrane 80, proximate the counter electrode element 68. As described above, the electrolyte 72 may provide ions or donate charges to prevent or inhibit the formation of gas bubbles (e.g., hydrogen or oxygen, depending on the polarity of the electrode) on the counter electrode element 68 and may prevent or inhibit the formation of acids or bases or neutralize the same, which may enhance efficiency and/or reduce the potential for irritation of the biological interface 18.

The inner ion selective membrane 74 may be positioned between the electrolyte 72 and the buffer material 78. The inner ion selective membrane 74 may take the form of a charge selective membrane, such as the illustrated ion exchange membrane that substantially allows passage of ions of a first polarity or charge while substantially blocking passage of ions or charge of a second, opposite polarity. The inner ion selective membrane 74 will typically pass ions of opposite polarity or charge to those passed by the outermost ion selective membrane 80 while substantially blocking ions of like polarity or charge. Alternatively, the inner ion selective membrane 74 may take the form of a semi-permeable or microporous membrane that is selective based on size.

The inner ion selective membrane 74 may prevent transfer of undesirable elements or compounds into the buffer material 78. For example, the inner ion selective membrane 74 may prevent or inhibit the transfer of hydroxyl (OH⁻) or chloride (C⁻) ions from the electrolyte 72 into the buffer material 78.

The optional buffer reservoir 76 is generally disposed between the electrolyte reservoir and the outermost ion selective membrane 80. The buffer reservoir 76 may take a variety of forms capable of temporarily retaining the buffer material 78. For example, the buffer reservoir 76 may take the form of a cavity, a porous membrane, or a gel. The buffer material 78 may supply ions for transfer through the outermost ion selective membrane 42 to the biological interface 18. Consequently, the buffer material 78 may comprise, for example, a salt (e.g., NaCl).

The outermost ion selective membrane 80 of the counter electrode assembly 14 may take a variety of forms. For example, the outermost ion selective membrane 80 may take the form of a charge selective ion exchange membrane. Typically, the outermost ion selective membrane 80 of the counter electrode assembly 14 is selective to ions with a charge or polarity opposite to that of the outermost ion selective membrane 38 of the active electrode assembly 12. The outermost ion selective membrane 80 is therefore an anion exchange membrane, which substantially passes anions and blocks cations, thereby prevents the back flux of the cations from the biological interface. Examples of suitable ion exchange membranes include the previously discussed membranes.

Alternatively, the outermost ion selective membrane 80 may take the form of a semi-permeable membrane that substantially passes and/or blocks ions based on size or molecular weight of the ion.

In any of the embodiments above, reservoirs or membranes may optionally comprise a dendritic polymer or arborol or gels or matrices assembled therefrom, as further described elsewhere herein.

The outer release liner 82 may generally be positioned overlying or covering an outer surface 84 of the outermost ion selective membrane 80. Note that the outer release liner 82 is shown in place in FIG. 2A and removed in FIG. 2B. The outer release liner 82 may protect the outermost ion selective membrane 80 during storage, prior to application of an electromotive force or current. The outer release liner 82 may be a selectively releasable liner made of waterproof material, such as release liners commonly associated with pressure sensitive adhesives. In some embodiments, the outer release liner 82 may be coextensive with the outer release liner 46 of the active electrode assembly 12.

The transdermal delivery system 10 may further comprise an inert molding material 86 adjacent exposed sides of the various other structures forming the active and counter electrode assemblies 12,14. The molding material 86 may advantageously provide environmental protection to the various structures of the active and counter electrode assemblies 12,14. Enveloping the active and counter electrode assemblies 12,14 is a housing material 90.

As best seen in FIG. 2B, the active and counter electrode assemblies 12, 14 are positioned on the biological interface 18. Positioning on the biological interface may close the circuit, allowing electromotive force to be applied and/or current to flow from the power source 16 to the active electrode assembly, to the biological interface 18, and to the counter electrode assembly 14.

In use, the outermost active electrode ion selective membrane 38 may be placed directly in contact with the biological interface 18. Alternatively, an interface-coupling medium (not shown) may be employed between the outermost active electrode ion selective membrane 22 and the biological interface 18. The interface-coupling medium may take, for example, the form of an adhesive and/or gel. The gel may take, for example, the form of a hydrating gel or a hydrogel. If used, the interface-coupling medium should be permeable by the active agent 36, 40, 42.

The power source 16 may take, for example, the form of one or more chemical battery cells, super- or ultra-capacitors, fuel cells, secondary cells, thin film secondary cells, button cells, lithium ion cells, zinc air cells, nickel metal hydride cells, and the like. In some embodiments, the power source 16 is selected to provide sufficient voltage, current, and/or duration to ensure delivery of the one or more active agents 36, 40, 42 from the reservoir 34 and across a biological interface (e.g., a skin or mucous membrane) to impart a desired physiological effect. The power source 16 may, for example, provide a voltage of 12.8V DC, with tolerance of 0.8V DC, and a current of 0.3 mA. The power source 16 may be selectively electrically coupled to the active and counter electrode assemblies 12, 14 via a control circuit, for example, via carbon fiber ribbons. The transdermal delivery system 10 may include discrete and/or integrated circuit elements to control the voltage, current, and/or power delivered to the electrode assemblies 12, 14. For example, the transdermal delivery system 10 may include a diode to provide a constant current to the electrode elements 24, 68.

As suggested above, the one or more active agents 36, 40, 42 may take the form of one or more ionic, ionizable, and/or neutral drugs or other therapeutic or diagnostic agents. Consequently, the poles or terminals of the power source 16 and the selectivity of the outermost ion selective membranes 38, 80 and inner ion selective membranes 30, 74 are selected accordingly.

During iontophoresis, the electromotive force across the electrode assemblies, as described, leads to a migration of charged active agent molecules, as well as ions and other charged components, through the biological interface into the biological tissue. This migration may lead to an accumulation of active agents, ions, and/or other charged components within the biological tissue beyond the interface. During iontophoresis, in addition to the migration of charged molecules in response to repulsive forces, there is also an electroosmotic flow of solvent (e.g., water) through the electrodes and the biological interface into the tissue. In certain embodiments, the electroosmotic solvent flow enhances migration of both charged and uncharged molecules. Enhanced migration via electroosmotic solvent flow may occur particularly with increasing size of the molecule.

In certain embodiments, the active agent may be a higher molecular weight molecule. In certain aspects, the molecule may be a polar polyelectrolyte. In certain other aspects, the molecule may be lipophilic. In certain embodiments, such molecules may be charged, may have a low net charge, or may be uncharged under the conditions within the active electrode. In certain aspects, active agents may migrate poorly under the iontophoretic repulsive forces, in contrast to the migration of small more highly charged active agents under the influence of these forces. These higher molecular weight active agents may thus be carried through the biological interface into the underlying tissues primarily via electroosmotic solvent flow. In certain embodiments, the high molecular weight polyelectrolytic active agents may be proteins, polypeptides, or nucleic acids. In other embodiments, the active agent may be mixed with another agent to form a complex capable of being transported across a biological interface via one of the motive methods described above.

The above description of illustrated embodiments, including what is described in the Abstract, is not intended to be exhaustive or to limit the claims to the precise forms disclosed. Although specific embodiments and examples are described herein for illustrative purposes, various equivalent modifications can be made without departing from the spirit and scope of the invention, as will be recognized by those skilled in the relevant art. The teachings provided herein can be applied to other agent delivery systems and devices, not necessarily the exemplary iontophoresis active agent system and devices generally described above. For instance, some embodiments may omit one or more reservoirs, membranes or other structure. In other instances, some embodiments may include additional structure. For example, some embodiment may include a control circuit or subsystem to control a voltage, current or power applied to the active and counter electrode elements 24, 68. Also for example, some embodiments may include an interface layer interposed between the outermost active electrode ion selective membrane 38 and the biological interface 18. Some embodiments may comprise additional ion selective membranes, ion exchange membranes, semi-permeable membranes and/or porous membranes, as well as additional reservoirs for electrolytes and/or buffers.

Various electrically conductive hydrogels, in addition to those comprising dendritic polymers or arborols, including those self-assembled from such materials, as described elsewhere herein, have been known and used in the medical field to provide an electrical interface to the skin of a subject or within a device to couple electrical stimulus into the subject. Hydrogels hydrate the skin, thus protecting against burning due to electrical stimulation through the hydrogel, while swelling the skin and allowing more efficient transfer of an active component. Examples of such hydrogels are disclosed in U.S. Pat. Nos. 6,803,420; 6,576,712; 6,908,681; 6,596,401; 6,329,488; 6,197,324; 5,290,585; 6,797,276; 5,800,685; 5,660,178; 5,573,668; 5,536,768; 5,489,624; 5,362,420; 5,338,490; and 5,240,995, herein incorporated in their entirety by reference. Further examples of such hydrogels are disclosed in U.S. Patent applications 2004/166147; 2004/105834; and 2004/247655, herein incorporated in their entirety by reference. Product brand names of various hydrogels and hydrogel sheets include Corplex™ by Corium, Tegagel™ by 3M, PuraMatrix™ by BD; Vigilon™ by Bard; ClearSite™ by Conmed Corporation; FlexiGel™ by Smith & Nephew; Derma-Gel™ by Medline; Nu-Gel™ by Johnson & Johnson; and Curagel™ by Kendall, or acrylhydrogel films available from Sun Contact Lens Co., Ltd.

In certain embodiments, preparations of dendrimers or arborols, or self-associating forms thereof, or various hydrogels described above may be made to incorporate proteins or polypeptides, or fusion proteins or fusion polypeptides, for use with devices and methods disclosed herein. In certain embodiments, such preparations may serve as reservoirs for the various active agents. Such preparations may constitute, for example, inner active agent reservoir 34 or layer 52 of the active electrode assembly in FIGS. 2A and 2B.

Various embodiments discussed herein may advantageously employ microstructures, for example, microneedles. Microneedles and microneedle arrays, their manufacture, and use have been described. Microneedles, either individually or in arrays, may be hollow; solid and permeable; solid and semi-permeable; or solid and non-permeable. Solid, non-permeable microneedles may further comprise grooves along their outer surfaces. Microneedles and microneedle arrays may be manufactured from a variety of materials, including silicon; silicon dioxide; molded plastic materials, including biodegradable or non-biodegradable polymers; ceramics; and metals. Microneedles, either individually or in arrays, may be used to dispense or sample fluids through the hollow apertures, through the solid permeable or semi-permeable materials, or via the external grooves. Microneedle devices may be used, for example, to deliver any of a variety of compounds and/or compositions to the living body via a biological interface, such as skin or mucous membrane. In certain embodiments, the active agent compounds and compositions may be delivered into or through the biological interface. For example, in delivering compounds or compositions via the skin, the length of the microneedle(s), either individually or in arrays, and/or the depth of insertion may be used to control whether administration of a compound or composition is only into the epidermis, through the epidermis to the dermis, or subcutaneous. In certain embodiments, microneedle devices may be useful for delivery of high-molecular weight active agents, such as those comprising proteins, peptides and/or nucleic acids, and corresponding compositions thereof. In certain embodiments, for example wherein the fluid is an ionic solution, microneedle(s) or microneedle array(s) can provide electrical continuity between a power source and the tip of the microneedle(s). Microneedle(s) or microneedle array(s) may be used advantageously to deliver or sample compounds or compositions by iontophoretic methods, as disclosed herein. In certain embodiments, for example, a plurality of microneedles in an array may advantageously be formed on an outermost biological interface-contacting surface of an iontophoresis device.

Certain details of microneedle devices, their use and manufacture, are disclosed in U.S. Pat. Nos. 6,256,533; 6,312,612; 6,334,856; 6,379,324; 6,451,240; 6,471,903; 6,503,231; 6,511,463; 6,533,949; 6,565,532; 6,603,987; 6,611,707; 6,663,820; 6,767,341; 6,790,372; 6,815,360; 6,881,203; 6,908,453; and 6,939,311; all of which are incorporated herein by reference in their entirety. Some or all of the teaching therein may be applied to microneedle devices, their manufacture, and their use in iontophoretic applications.

In certain embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the power source; an active agent reservoir having a solution of an active agent, such as a drug or therapeutic or diagnostic agent, that is in contact with the first electrode member and to which is applied a voltage via the first electrode member; a biological interface contact member, which may be a microneedle array and is placed against the forward surface of the active agent reservoir; and a first cover or container that accommodates these members. The counter electrode assembly includes the following: a second electrode member connected to a negative electrode of the voltage source; a second electrolyte reservoir that holds an electrolyte that is in contact with the second electrode member and to which voltage is applied via the second electrode member; and a second cover or container that accommodates these members.

In certain other embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the voltage source; a first electrolyte reservoir having an electrolyte that is in contact with the first electrode member and to which is applied a voltage via the first electrode member; a first anion-exchange membrane that is placed on the forward surface of the first electrolyte reservoir; an active agent reservoir that is placed against the forward surface of the first anion-exchange membrane; a biological interface contacting member, which may be a microneedle array and is placed against the forward surface of the active agent reservoir; and a first cover or container that accommodates these members. The counter electrode assembly includes the following: a second electrode member connected to a negative electrode of the voltage source; a second electrolyte reservoir having an electrolyte that is in contact with the second electrode member and to which is applied a voltage via the second electrode member; a cation-exchange membrane that is placed on the forward surface of the second electrolyte reservoir; a third electrolyte reservoir that is placed against the forward surface of the cation-exchange membrane and holds an electrolyte to which a voltage is applied from the second electrode member via the second electrolyte reservoir and the cation-exchange membrane; a second anion-exchange membrane placed against the forward surface of the third electrolyte reservoir; and a second cover or container that accommodates these members.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety, including but not limited to: Japanese patent application Serial No. H03-86002, filed Mar. 27, 1991, having Japanese Publication No. H04-297277, issued on Mar. 3, 2000 as Japanese Patent No. 3040517; Japanese patent application Serial No. 11-033076, filed Feb. 10, 1999, having Japanese Publication No. 2000-229128; Japanese patent application Serial No. 11-033765, filed Feb. 12, 1999, having Japanese Publication No. 2000-229129; Japanese patent application Serial No. 11-041415, filed Feb. 19, 1999, having Japanese Publication No. 2000-237326; Japanese patent application Serial No. 11-041416, filed Feb. 19, 1999, having Japanese Publication No. 2000-237327; Japanese patent application Serial No. 11-042752, filed Feb. 22, 1999, having Japanese Publication No. 2000-237328; Japanese patent application Serial No. 11-042753, filed Feb. 22, 1999, having Japanese Publication No. 2000-237329; Japanese patent application Serial No. 11-099008, filed Apr. 6, 1999, having Japanese Publication No. 2000-288098; Japanese patent application Serial No. 11-099009, filed Apr. 6, 1999, having Japanese Publication No. 2000-288097; PCT patent application WO 2002JP4696, filed May 15, 2002, having PCT Publication No WO03037425; U.S. patent application Ser. No. 10/488,970, filed Mar. 9, 2004; Japanese patent application 2004/317317, filed Oct. 29, 2004; U.S. provisional patent application Ser. No. 60/627,952, filed Nov. 16, 2004; Japanese patent application Serial No. 2004-347814, filed Nov. 30, 2004; Japanese patent application Serial No. 2004-357313, filed Dec. 9, 2004; Japanese patent application Serial No. 2005-027748, filed Feb. 3, 2005; and Japanese patent application Serial No. 2005-081220, filed Mar. 22, 2005.

As one skilled in the relevant art would readily appreciate, the present disclosure comprises methods of treating a subject by any of the compositions and/or methods described herein.

Aspects of the various embodiments can be modified, if necessary, to employ systems, circuits and concepts of the various patents, applications and publications to provide yet further embodiments, including those patents and applications identified herein. While some embodiments may include all of the membranes, reservoirs and other structures discussed above, other embodiments may omit some of the membranes, reservoirs or other structures. Still other embodiments may employ additional ones of the membranes, reservoirs and structures generally described above. Even further embodiments may omit some of the membranes, reservoirs and structures described above while employing additional ones of the membranes, reservoirs and structures generally described above.

These and other changes can be made in light of the above-detailed description. In general, in the following claims, the terms used should not be construed to be limiting to the specific embodiments disclosed in the specification and the claims, but should be construed to include all systems, devices and/or methods that operate in accordance with the claims. Accordingly, the invention is not limited by the disclosure, but instead its scope is to be determined entirely by the following claims.

We/I claim:

1. A device for delivery of one or more active agents to a biological interface, comprising:
a matrix comprising at least one dendritic polymer or arborol; and
at least one active agent.

2. The device of claim 1 wherein the device is a transdermal delivery device.

3. The device of claim 1 wherein the device is an iontophoretic device having at least two electrode structures.

4. The device of claim 1 wherein the arborol is a one-directional arborol.

5. The device of claim 4 wherein the one-directional arborol is a [9]-6 arborol, a [9]-8 arborol, or a [9]-10 arborol.

6. The device of claim 1 wherein the arborol is a two-directional arborol.

7. The device of claim 6 wherein the two-directional arborol is a [m]-n-[m] arborol wherein m is the number of polar groups comprising each of two hydrophilic end regions and n is the number of carbons in a linear alkyl chain connecting the two hydrophilic end regions.

8. The device of claim 6 wherein the two-directional arborol is a [9]-n-[9] arborol wherein n is the number of carbons in a linear alkyl chain connecting two hydrophilic end regions each comprising 9 hydroxyl groups.

9. The device of claim 8 wherein n is 10-13.

10. The device of claim 9 wherein the two-directional arborol is a [9]-10-[9] arborol.

11. The device of claim 1 wherein the arborol is a three-directional arborol.

12. The device of claim 11 wherein the three-directional arborol is benzene[9³].

13. The device of claim 1 wherein the dendritic polymer is polyamidoamine (PAMAM) dendrimer.

14. The device of claim 1 wherein the dendritic polymer is a polypropylene imine (PPI) dendrimer.

15. The device of claim 1 wherein the dendritic polymer is a polyether or phenylacetylene dendrimer.

16. The device of claim 1 wherein the matrix is a gel matrix.

17. The device of claim 1 wherein the dendritic polymer or arborol is a self-assembling dendritic polymer or arborol.

18. The device of claim 17 wherein the self-assembling dendritic polymer or arborol is a two-directional arborol.

19. The device of claim 18 wherein the two-directional arborol is a [m]-n-[m] arborol wherein m is the number of polar groups comprising each of two hydrophilic end regions and n is the number of carbons in a linear alkyl chain connecting the two hydrophilic end regions.

20. The device of claim 19 wherein the two-directional arborol is a [9]-n-[9] arborol wherein n is the number of carbons in a linear alkyl chain connecting two hydrophilic end regions each comprising 9 hydroxyl groups.

21. The device of claim 20 wherein n is 10-13.

22. The device of claim 21 wherein the two-directional arborol is a [9]-10-[9] arborol.

23. The device of claim 1 wherein the active agent is a therapeutic agent, a diagnostic agent, or a pharmaceutical drug.

24. The device of claim 1, further comprising:
at least one reservoir.

25. The device of claim 1 wherein the matrix comprising at least one dendritic polymer or arborol forms a reservoir.

26. The device of claim 25 wherein the dendritic polymer or arborol is a self-assembling dendritic polymer or arborol.

27. The device of claim 1, further comprising:
at least one membrane.

28. The device of claim 1 wherein the matrix comprising at least one dendritic polymer or arborol forms at least one membrane.

29. The device of claim 28 wherein the dendritic polymer or arborol is a self-assembling dendritic polymer or arborol.

30. The device of claim 29 wherein self-assembling the dendritic polymer or arborol forms at least one membrane.

31. The device of claim 1 wherein the dendritic polymer or arborol comprises a charged group.

32. The device of claim 31 wherein the charged group has a net positive charge.

33. The device of claim 31 wherein the charged group has a net negative charge.

34. The device of claim 1, further comprising:
an interface-coupling medium between a surface of the device and the biological interface.

35. The device of claim 34 wherein the interface-coupling medium is an adhesive.

36. A device for delivery of one or more active agents to a biological interface, comprising:
a matrix comprising at least one dendritic polymer or arborol; and
an electrode structure.

37. The device of claim 36 wherein the device is a transdermal delivery device.

38. The device of claim 36 wherein the device is an iontophoretic device having at least two electrode structures.

39. The device of claim 36 wherein the arborol is a one-directional arborol.

40. The device of claim 36 wherein the arborol is a two-directional arborol.

41. The device of claim 36 wherein the arborol is a three-directional arborol.

42. The device of claim 36 wherein the dendritic polymer is polyamidoamine (PAMAM) dendrimer.

43. The device of claim 36 wherein the dendritic polymer is a polypropylene imine (PPI) dendrimer.

44. The device of claim 36 wherein the dendritic polymer is a polyether or phenylacetylene dendrimer.

45. The device of claim 36 wherein the matrix is a gel matrix.

46. The device of claim 36 wherein the dendritic polymer or arborol is a self-assembling dendritic polymer or arborol.

47. The device of claim 46 wherein the self-assembling dendritic polymer or arborol is a two-directional arborol.

48. The device of claim 47 wherein the two-directional arborol is a [m]-n-[m] arborol wherein m is the number of polar groups comprising each of two hydrophilic end regions and n is the number of carbons in a linear alkyl chain connecting the two hydrophilic end regions.

49. The device of claim 36 wherein the active agent is a therapeutic agent, diagnostic agent, or a pharmaceutical drug.

50. The device of claim 36, further comprising:
at least one reservoir comprising a dendritic polymer or arborol.

51. The device of claim 36, further comprising:
at least one membrane comprising a dendritic polymer or arborol.

52. The device of claim 36 wherein the dendritic polymer or arborol comprises a charged group.

53. A method for making an active agent delivery device, comprising:
placing in a portion of the device a solution or suspension of a self-associating dendritic polymer; and
allowing the self-associating dendritic polymer to self associate.

54. The method of claim 53 wherein the portion of the device is a reservoir.

55. The method of claim 53 wherein the portion of the device is a membrane.

56. The method of claim 53 wherein the self-associating dendritic polymer is an arborol.

57. The method of claim 53, further comprising: loading an active agent into pores or cavities created by self-association of the self-associating dendritic polymer.

58. An active agent delivery device made according to the method of any one of claims 53-57.
